(19) United States
(12) Patent Application Publication
(10) Pub. No.: US 2022/0338777 A1
(43) Pub. Date: Oct. 27, 2022

(54) BIOPOTENTIAL MEASURING APPARATUS, BIOPOTENTIAL MEASURING SYSTEM, AND BIOPOTENTIAL MEASURING METHOD

(71) Applicant: SONY GROUP CORPORATION, TOKYO (JP)

(72) Inventor: KAZUNARI YOSHIFUJI, TOKYO (US)

(21) Appl. No.: 17/753,743

(22) PCT Filed: Sep. 15, 2020
(86) PCT No.: PCT/JP2020/034914

§ 371 Date: Mar. 14, 2022

(30) Foreign Application Priority Data
Sep. 24, 2019 (JP) 2019-172957

(51) Int. Cl. A61B 5/273

(52) U.S. Cl. CPC A61B 5/273

(57) ABSTRACT

[Object] Proposed are a biopotential measuring apparatus, a biopotential measuring system, and a biopotential measuring method that are capable of suppressing an erroneous determination in determining the quality of a contact state between an electrode and a living body. A biopotential measuring apparatus of the present technology includes an electrode and a controller. The electrode measures a biopotential. The controller determines a quality of a contact state between the electrode and a living body on the basis of a signal amplitude of a signal of a specific frequency.

### TECHNICAL FIELD

The present technology relates to a biopotential measuring apparatus, a biopotential measuring system, and a biopotential measuring method.

### BACKGROUND ART

An apparatus that acquires biological information such as an electroencephalogram or an electrocardiogram by measuring contact impedance of an electrode being in contact with the skin of a user has been conventionally known as a biopotential measuring apparatus (for example, Patent Literature 1).

### CITATION LIST

### Patent Literature

Patent Literature 1: Japanese Patent Application Laid-open No. 2018-99283

### DISCLOSURE OF INVENTION

### Technical Problem

However, in the conventional biopotential measuring apparatus, it is impossible to accurately determine whether the contact state between the electrode and the skin of the user is good or not depending on the wearing state of the user, which may cause an erroneous determination.

In this regard, the present disclosure proposes a biopotential measuring apparatus, a biopotential measuring system, and a biopotential measuring method that are capable of suppressing an erroneous determination in determining the quality of a contact state between an electrode and a living body.

### Solution to Problem

In order to solve the above problem, a biopotential measuring apparatus according to an embodiment of the present technology includes an electrode and a controller.

The electrode measures a biopotential.

The controller determines a quality of a contact state between the electrode and a living body on the basis of a signal amplitude of a signal of a specific frequency.

The controller may determine the quality of the contact state on the basis of whether the signal amplitude exceeds a predetermined threshold value.

The signal of the specific frequency may be a signal resulting from a commercial power supply.

The biopotential measuring apparatus may further include a reference electrode that provides a reference point of a potential at a time when a potential of the electrode is measured.

The biopotential measuring apparatus may further include an amplifier circuit that amplifies a potential difference between a potential measured by the electrode and a potential measured by the reference electrode.

The electrode may include a first measurement electrode and a second measurement electrode, and the amplifier circuit may include a first amplifier circuit connected to the first measurement electrode, and a second amplifier circuit connected to the second measurement electrode.

The controller may determine that a contact state between the living body and the reference electrode is poor when each of a signal amplitude of a signal resulting from the commercial power supply and output from the first amplifier circuit and a signal amplitude of a signal resulting from the commercial power supply and output from the second amplifier circuit exceeds a predetermined threshold value.

The controller may determine that a contact state between the living body and the reference electrode is good when one of a signal amplitude of a signal resulting from the commercial power supply and output from the first amplifier circuit and a signal amplitude of a signal resulting from the commercial power supply and output from the second amplifier circuit does not exceed a predetermined threshold value.

The controller may determine that a contact state between the first measurement electrode and the living body is poor and that a contact state between the second measurement electrode and the living body is good when the signal amplitude of the signal resulting from the commercial power supply and output from the first amplifier circuit exceeds the predetermined threshold value.

The controller may determine that the contact state between the first measurement electrode and the living body is good and that the contact state between the second measurement electrode and the living body is poor when the signal amplitude of the signal resulting from the commercial power supply and output from the first amplifier circuit does not exceed the predetermined threshold value and when the signal amplitude of the signal resulting from the commercial power supply and output from the second amplifier circuit exceeds the predetermined threshold value.

The controller may determine that the contact state between each of the first and second measurement electrodes and the living body is good when each of the signal amplitude of the signal resulting from the commercial power supply and output from the first amplifier circuit and the signal amplitude of the signal resulting from the commercial power supply and output from the second amplifier circuit does not exceed the predetermined threshold value.

The controller may cause a display apparatus to display information for making a user confirm the contact state when determining that the contact state is poor.

The biopotential measuring apparatus may be configured to be capable of measuring an electroencephalogram.

The biopotential measuring apparatus may be configured to be capable of measuring an electrocardiogram.

In order to solve the above problem, a biopotential measuring system according to an embodiment of the present technology includes a biopotential measuring apparatus and a display apparatus.

The biopotential measuring apparatus includes an electrode and a controller.

The electrode measures a biopotential.

The controller determines a quality of a contact state between the electrode and a living body on the basis of a signal amplitude of a signal of a specific frequency.

The display apparatus displays the biopotential and the contact state between the electrode and the living body.

The display apparatus may display information for making a user confirm the contact state when the controller determines that the contact state is poor.

In order to solve the above problem, a biopotential measuring method for a biopotential measuring apparatus according to an embodiment of the present technology includes: measuring a biopotential; and determining a quality of a contact state between the electrode and a living body on the basis of a signal amplitude of a signal of a specific frequency.

### BRIEF DESCRIPTION OF DRAWINGS

FIG. 1 is a schematic diagram showing a configuration example of a biopotential measuring system according to this embodiment.

FIG. 2 is a diagram showing a detailed configuration of a biopotential measuring apparatus of the biopotential measuring system.

FIG. 3 is a block diagram showing a hardware configuration example of the biopotential measuring apparatus and an information processing apparatus of the biopotential measuring system.

FIG. 4 is a flowchart showing a typical operation flow of the biopotential measuring apparatus.

FIG. 5 is a diagram showing a detailed configuration of the biopotential measuring apparatus.

FIG. 6 is a diagram showing examples of a display screen of a display apparatus of the biopotential measuring system.

FIG. 7 is a diagram showing a detailed configuration of the biopotential measuring apparatus.

FIG. 8 is a diagram showing a detailed configuration of the biopotential measuring apparatus.

FIG. 9 is a diagram showing a detailed configuration of the biopotential measuring apparatus.

### MODE(S) FOR CARRYING OUT THE INVENTION

Hereinafter, an embodiment of the present technology will be described with reference to the drawings.

<Configuration of Biopotential Measuring System>

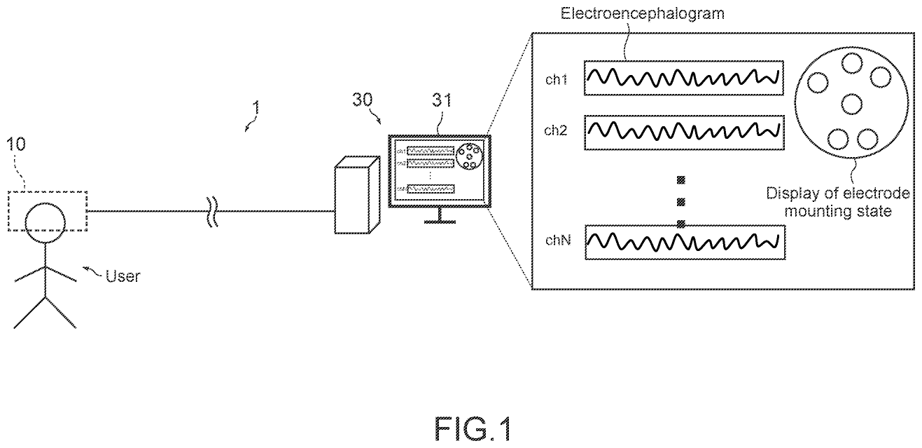

FIG. 1 is a schematic diagram showing a configuration example of a biopotential measuring system 1 of this embodiment. As shown in FIG. 1, the biopotential measuring system 1 includes a biopotential measuring apparatus 10, an information processing apparatus 30, and a display apparatus 31.

The biopotential measuring apparatus 10 and the information processing apparatus 30 are connected to each other by wireless or wired connection. Alternatively, the biopotential measuring apparatus 10 and the information processing apparatus 30 may be connected to each other via any network so as to be able to communicate with each other. In this case, the network may be the Internet, a mobile communication network, a local area network, or the like, and may be a network in which a plurality of types of networks is combined.

[Biopotential Measuring Apparatus]

The biopotential measuring apparatus 10 is typically an electroencephalograph headset that is mounted on the head of a user. The biopotential measuring apparatus 10 includes a plurality of measurement electrodes for measuring a biopotential, and a reference electrode serving as a reference for obtaining a potential difference from the potential measured by the measurement electrodes.

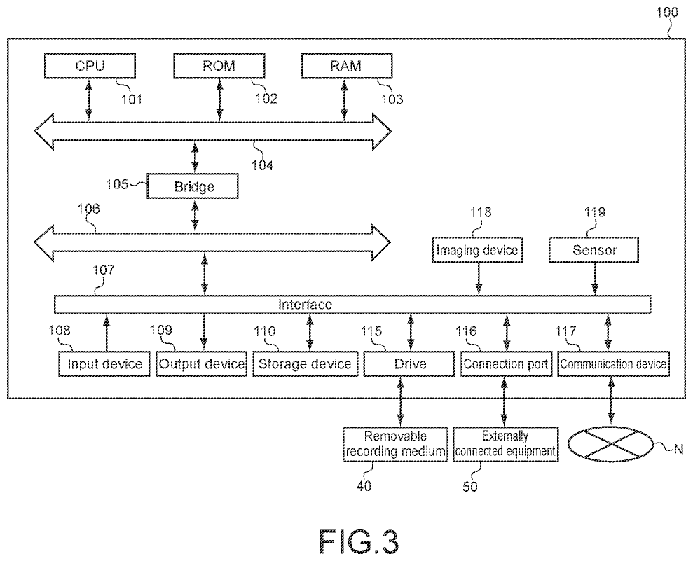

The biopotential measuring apparatus 10 includes an input device (see FIG. 3) for inputting operation information for a user to operate the biopotential measuring system 1, and operation information for implementing an operation desired by the user is input thereto.

The biopotential measuring apparatus 10 measures the biopotential of the user. Information regarding the biopotential measured by the biopotential measuring apparatus 10 is output to the information processing apparatus 30.

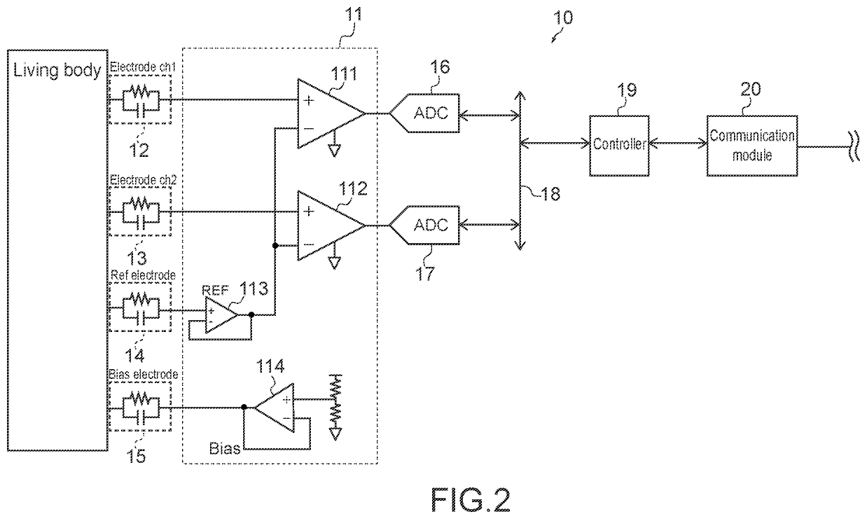

FIG. 2 is a diagram showing a detailed configuration of a main part of the biopotential measuring apparatus 10. The biopotential measuring apparatus 10 includes a differential amplifier circuit 11, a first measurement electrode 12, a second measurement electrode 13, a reference electrode 14, a bias electrode 15, ADCs 16 and 17, a bus 18, a controller 19, and a communication module 20. The differential amplifier circuit 11 is an example of an “amplifier circuit” in the Claims.

As shown in FIG. 2, the differential amplifier circuit 11 includes amplifier circuits 111 and 112 and impedance conversion circuits 113 and 114. The amplifier circuit 111 is an amplifier circuit that amplifies a biopotential (electroencephalogram) corresponding to a potential difference between the first measurement electrode 12 and the reference electrode 14.

The positive input terminal of the amplifier circuit 111 is connected to the first measurement electrode 12. The negative input terminal of the amplifier circuit 111 is connected to the output terminal of the impedance conversion circuit 113. The output of the differential amplifier 111 is connected to the ADC 16.

The amplifier circuit 112 is an amplifier circuit that amplifies a biopotential corresponding to a potential difference between the second measurement electrode 13 and the reference electrode 14. The amplifier circuit 112 measures the potential difference between the second measurement electrode 13 and the reference electrode 14 and amplifies the measured potential difference.

The positive input terminal of the amplifier circuit 112 is connected to the second measurement electrode 13. The negative input terminal of the amplifier circuit 112 is connected to the output terminal of the amplifier circuit 112. The output of the amplifier circuit 112 is connected to the ADC 17.

The impedance conversion circuit 113 is a circuit that does not amplify the potential measured by the reference electrode 14 and that converts the impedance. The impedance circuit 113 is a so-called voltage follower circuit. The positive input terminal of the amplifier circuit 113 is connected to the reference electrode 14. The output of the differential amplifier 113 is connected to the negative input terminals of the differential amplifiers 111 and 112.

The impedance conversion circuit 114 is a circuit that applies a fixed potential of a resistance voltage divider connected to the positive input terminal to the living body, and is a circuit referred to as a voltage follower circuit similarly to the impedance conversion circuit 113. The impedance conversion circuit 114 is capable of applying a fixed potential to the living body, regardless of the impedance between the biopotential measuring apparatus 10 and the living body. The output of the impedance conversion circuit 114 is connected to the bias electrode 15.

The first and second measurement electrodes 12 and 13 measure the electroencephalogram of the user. The reference electrode 14 is a reference electrode for providing a reference point of a potential at the time when the first and second measurement electrodes 12 and 13 measure a biopotential. The bias electrode 15 is an electrode that determines the potential relationship between the biopotential measuring apparatus 10 and the living body.

The first and second measurement electrodes 12 and 13, the reference electrode 14, and the bias electrode 15 are electrodes for acquiring a potential from the living body. Those electrodes 12 to 15 are typically, but not limited to, Ag/AgCl electrodes, and may be formed of, for example, gold (Au) or stainless steel. Further, the first and second measurement electrodes 12 and 13, the reference electrode 14, and the bias electrode 15 may be gel electrodes, dry electrodes, or wet electrodes.

The ADC 16 is an A/D converter that converts the analog signal of the potential difference amplified by the amplifier circuit 111 into a digital signal and outputs the digital signal to the controller 19 via the bus 18. Similarly, the ADC 17 is an A/D converter that converts the analog signal of the potential difference amplified by the amplifier circuit 112 into a digital signal and outputs the digital signal to the controller 19 via the bus 18.

The controller 19 controls the overall operation of the biopotential measuring apparatus 10 or part thereof according to programs. Specifically, the controller 19 controls timings at which the digital value for the ADC 16 and the digital value for ADC 17 are read and controls various measurement modes. The readout timings are usually performed at fixed intervals. For example, if the measurement frequency of the biopotential measurement is 1000 Hz, the readout of the ADC 16 and the ADC 17 is performed every 1 ms.

The communication module 20 communicates with the information processing apparatus 30. The communication module 20 functions as a communication interface of the biopotential measuring apparatus 10.

[Information Processing Apparatus]

The information processing apparatus 30 performs predetermined processing on the biopotential signal acquired by the biopotential measuring apparatus 10 and outputs the processing result to the display apparatus 31. The information processing apparatus 30 is connected to the biopotential measuring apparatus 10 in a wired or wireless manner.

The information processing apparatus 30 is typically, but not limited to, a desktop PC, and may be any other computer such as a laptop PC.

[Display Apparatus]

The display apparatus 31 displays the processing result processed by the information processing apparatus 30. The display apparatus 31 displays the measurement result of the biopotential measured by each of the measurement electrodes being in contact with the head of the user. Further, the display apparatus 31 displays the mounting state of each measurement electrode being in contact with the head of the user.

[Hardware Configuration]

FIG. 3 is a block diagram showing an example of a hardware configuration of the biopotential measuring apparatus 10 and the information processing apparatus 30. The biopotential measuring apparatus 10 and the information processing apparatus 30 may be an information processing apparatus 100 shown in FIG. 3.

The information processing apparatus 100 includes a central processing unit (CPU) 101, a read only memory (ROM) 012, and a random access memory (RAM) 103. Further, the information processing apparatus 100 may include a host bus 104, a bridge 105, an external bus 106, an interface 107, an input device 108, an output device 109, a storage device 110, a drive 115, a connection port 116, and a communication device 117.

Furthermore, the information processing apparatus 100 may include an imaging device 118 and a sensor 119 as needed. The information processing apparatus 100 may include a processing circuit such as a digital signal processor (DSP), an application specific integrated circuit (ASIC), or a field-programmable gate array (FPGA) instead of or together with the CPU 101.

The CPU 101 functions as an arithmetic processing unit and a control unit, and controls the overall operation of the information processing apparatus 100 or part thereof according to various programs recorded in the ROM 102, the RAM 103, the storage device 110, or on a removable recording medium 40. The controller 19 may be the CPU 101.

The ROM 102 stores programs to be used by the CPU 101, calculation parameters, and the like. The RAM 103 temporarily stores the programs to be used in the execution of the CPU 101, parameters that appropriately change in the execution of the programs, and the like.

The CPU 101, the ROM 102, and the RAM 103 are interconnected by a host bus 104 including an internal bus such as a CPU bus. In addition, the host bus 104 is connected via a bridge 105 to an external bus 106 such as a peripheral component interconnect/interface (PCI) bus.

The input device 108 is a device operated by a user, such as a mouse, a keyboard, a touch panel, a button, a switch, or a lever. The input device 108 may be, for example, a remote control device using infrared rays or other radio waves, or may be externally connected equipment 50 such as a mobile phone corresponding to the operation of the information processing apparatus 100.

The input device 108 includes input control circuits for generating input signals on the basis of information input by the user and outputting the generated input signals to the CPU 101. By operating the input device 108, the user inputs various types of data to the information processing apparatus 100 or instructs processing operations.

The output device 109 is configured by a device capable of notifying the user of the acquired information by using senses such as a sense of vision, a sense of hearing, and a sense of touch. The output device 109 may be, for example, a display apparatus such as a liquid crystal display (LCD) or an organic electro-luminescence (EL) display, a sound output device such as a speaker or headphones, or a vibrator.

The output device 109 outputs the result acquired by the processing of the information processing apparatus 100 as a video such as a text or an image, a sound such as voice or audio, or vibration. The display apparatus 31 corresponds to the output device 109.

The storage device 110 is a data storage device configured as an example of a storage unit of the information processing apparatus 100. The storage device 110 is configured by, for example, a magnetic storage device such as a hard disk drive (HDD), a semiconductor storage device, an optical storage device, or a magneto-optical storage device. The storage device 110 stores, for example, programs to be executed by the CPU 101, various types of data, and various types of data acquired from the outside.

The drive 115 is a reader/writer for the removable recording medium 40 such as a magnetic disk, an optical disc, a magneto-optical disk, or a semiconductor memory, and is built in or externally attached to the information processing apparatus 100. The drive 115 reads the information recorded on the removable recording medium 40 mounted thereon, and outputs the read information to the RAM 103. Further, the drive 115 writes a record on the removable recording medium 40 mounted thereon.

The connection port 116 is a port for connecting a device to the information processing apparatus 100. The connection port 116 may be, for example, a universal serial bus (USB) port, an IEEE1394 port, or a small computer system interface (SCSI) port. Further, the connection port 116 may be an RS-232C port, an optical audio terminal, a high-definition multimedia interface (HDMI) (registered trademark) port, or the like. The externally connected equipment 50 is connected to the connection port 116, and thus various types of data can be exchanged between the information processing apparatus 100 and the externally connected equipment 50.

The communication device 117 is, for example, a communication interface including a communication device for connecting to a communication network N. The communication device 117 may be, for example, a communication card for a local area network (LAN), Bluetooth (registered trademark), Wi-Fi, or wireless USB (WUSB).

The communication device 117 may be a router for optical communication, a router for asymmetric digital subscriber line (ADSL), or modems for various types of communication. The communication device 117 transmits and receives signals and the like to and from the Internet or other communication devices by using a predetermined protocol such as TCP/IP. The communication module 20 corresponds to the communication device 117.

Further, the communication network N connected to the communication device 117 is a network connected in a wired or wireless manner and may include, for example, the Internet, a home LAN, infrared communication, radio wave communication, and satellite communication.

The imaging device 118 is, for example, a device for capturing an image of a real space using an image pickup device such as a complementary metal oxide semiconductor (CMOS) or a charge coupled device (CCD) and various members such as a lens for controlling a subject image to be formed on the image pickup device, and then generating a captured image. The imaging device 118 may capture a still image or may capture a moving image.

The sensor 119 is, for example, various sensors such as an acceleration sensor, an angular velocity sensor, a geomagnetic sensor, an illuminance sensor, a temperature sensor, an atmospheric pressure sensor, and a sound sensor (microphone).

The sensor 119 acquires information regarding a state of the information processing apparatus 100, such as the posture of the housing of the information processing apparatus 100, and information regarding a surrounding environment of the information processing apparatus 100, such as brightness and noise around the information processing apparatus 100. Further, the sensor 119 may also include a global positioning system (GPS) receiver that receives GPS signals to measure the latitude, longitude, and altitude of the device.

An example of the configuration of the biopotential measuring system 1 has been described above. Each of the constituent components described above may be configured by using a general-purpose member or may be configured by hardware specialized for the function of each component. Such a configuration may be changed as appropriate according to the technical level at the time of implementation.

<Electrode Contact State Determining Method>

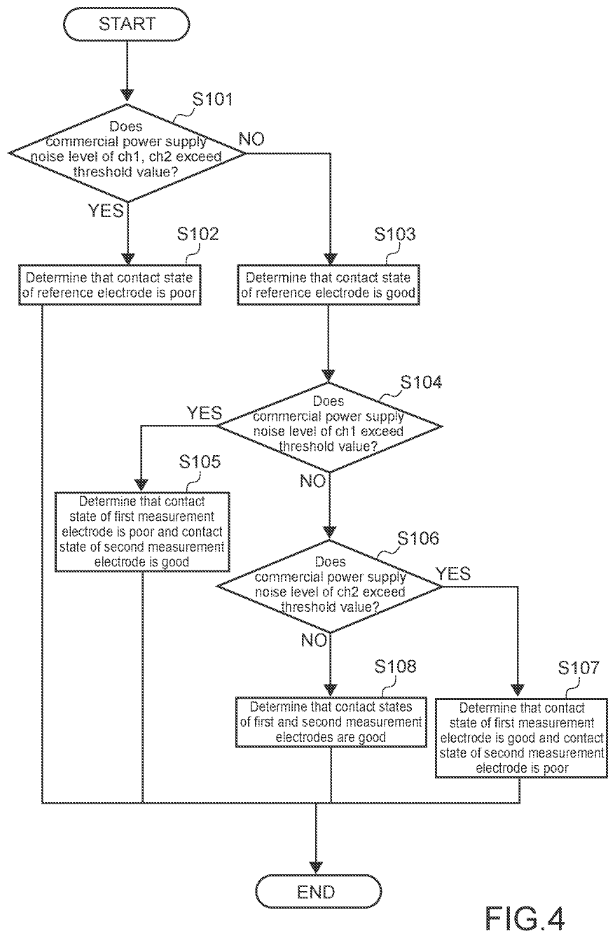

FIG. 4 is a flowchart showing a typical operation flow of the biopotential measuring apparatus 10. Hereinafter, a method of determining the quality of the contact state between the user and the electrode will be described with reference to FIG. 4 as appropriate.

First, before the operation of the biopotential measuring apparatus 10 of this embodiment is described, a commercial power supply noise will be described.

The commercial power supply noise in a local area where the measurement is performed propagates to the user whose electroencephalogram or the like is measured by the biopotential measuring apparatus 10. The frequency of the commercial power supply noise is 50 Hz in eastern Japan and 60 Hz in western Japan. The commercial power supply noise in this embodiment is a hum noise of 50 Hz, and the same applies to the following description.

In our surroundings, electric appliances that operate with an AC power supply of 100 V operate in many places in Japan, and commercial power supply noise derived from the AC power supply of 100 V propagates to reinforcing bars of buildings, metal furniture and fixtures in rooms, and the like. Under such circumstances, the noise propagates to a human body by capacitive coupling even if the person is not in physical contact with buildings or furniture and fixtures.

Thus, the commercial power supply noise is observed as common mode noise by the biopotential measuring apparatus 10. At that time, if the contact impedance matching between each of the measurement electrodes 12 and 13 and the reference electrode 14 is not obtained, the commercial power supply noise remains on the output side of the differential amplifier circuit 11.

In this regard, the biopotential measuring apparatus 10 of this embodiment determines the contact state of the first and second measurement electrodes 12 and 13 and the reference electrode 14 on the basis of whether the signal intensity of the commercial power supply noise remaining on the output side of the differential amplifier circuit 11 exceeds a predetermined threshold value or not. Hereinafter, some patterns of the contact state determining method will be described.

(Pattern 1)

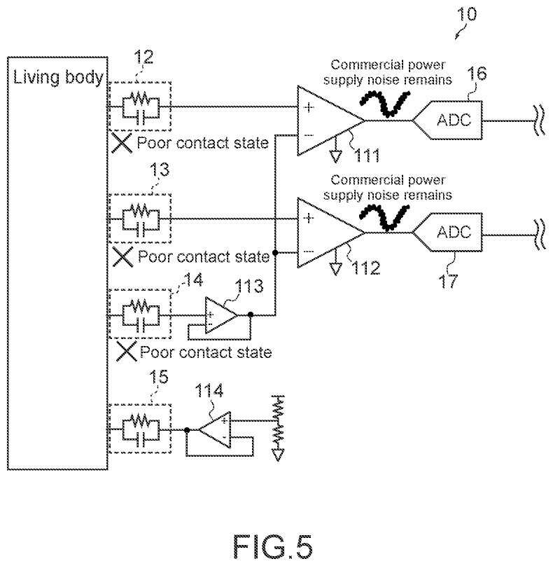

FIG. 5 is a diagram showing a detailed configuration of the biopotential measuring apparatus 10 and is a diagram showing a case where the contact state of the reference electrode 14 is poor. The controller 19 determines whether or not the signal amplitude of the commercial power supply noise remaining on the output side of the amplifier circuits 111 and 112 exceeds a predetermined threshold value (Step S101).

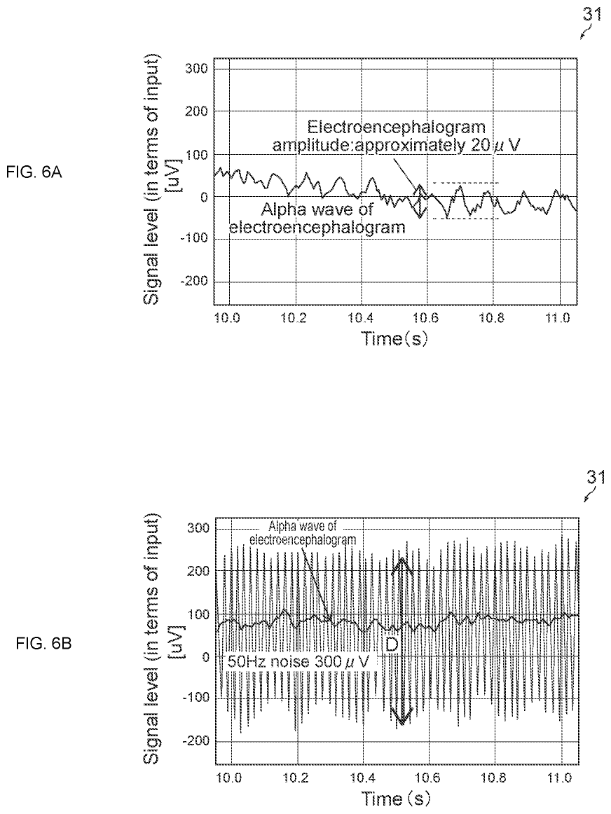

Specifically, the controller 19 determines whether or not a signal amplitude D of the commercial power supply noise remaining on the output side of the amplifier circuits 111 and 112 exceeds a predetermined threshold value when the user's electroencephalogram and the commercial power supply noise are displayed in a superimposed manner on the display apparatus 31 and observed as shown in FIG. 6b. When all of the signal amplitudes D of the noise exceed the predetermined threshold value (YES in Step S101), the controller 19 determines that the contact state between the reference electrode 14 and the user is poor (Step S102). FIG. 6 is a diagram showing examples of a display screen of the display apparatus 31.

If the controller 19 determines that the contact state between the reference electrode 14 and the user is poor, the display apparatus 31 displays information for prompting the user to confirm and improve the contact state between the reference electrode 14 and the user. Note that the predetermined threshold value may be arbitrarily set in accordance with the specification and the application of the biopotential measuring apparatus 10, and the like. This point applies to the patterns 2 to 4 to be described later.

(Pattern 2)

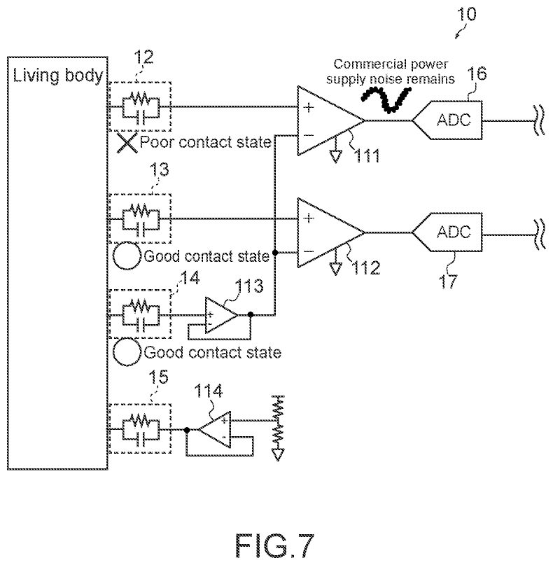

FIG. 7 is a diagram showing a detailed configuration of the biopotential measuring apparatus 10 and showing a case where the contact state of the first measurement electrode 12 is poor and the contact state of the first measurement electrode 12 and the reference electrode 14 is good.

If the signal amplitude D of any of the commercial power supply noise remaining on the output side of the amplifier circuits 111 and 112 does not exceed the predetermined threshold value (NO in Step S101), the controller 19 determines that the contact state between the reference electrode 14 and the user is good (Step S103).

Next, the controller 19 determines whether or not the signal amplitude D of the commercial power supply noise remaining on the output side of the amplifier circuit 111 exceeds the predetermined threshold value (Step S104). Here, if the signal amplitude D exceeds the predetermined threshold value (YES in Step S104), the controller 19 determines that the contact state between the first measurement electrode 12 and the user is poor, and determines that the contact state between the second measurement electrode 13 and the user is good (Step S105).

If the controller 19 determines that the contact state between the first measurement electrode 12 and the user is poor, the display apparatus 31 displays information prompting confirmation and improvement of the contact state between the first measurement electrode 12 and the user.

(Pattern 3)

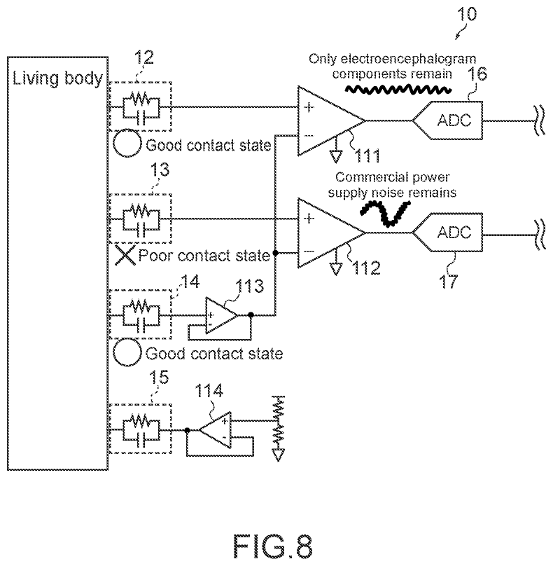

FIG. 8 is a diagram showing a detailed configuration of the biopotential measuring apparatus 10 and showing a case where the contact states of the first measurement electrode 12 and the reference electrode 14 are good and the contact state of the second measurement electrode 13 is poor.

If the signal amplitude D resulting from the commercial power supply and remaining on the output side of the amplifier circuit 111 does not exceed the predetermined threshold value (NO in Step S104), the controller 19 determines whether or not the signal amplitude D of the commercial power supply noise remaining on the output side of the amplifier circuit 112 exceeds the predetermined threshold value (Step S106). If the signal amplitude D exceeds the predetermined threshold value (YES in Step S106), the controller 19 determines that the contact state between the first measurement electrode 12 and the user is good, and determines that the contact state between the second measurement electrode 13 and the user is poor (Step S107).

If the controller 19 determines that the contact state between the second measurement electrode 13 and the user is poor, the display apparatus 31 displays information prompting confirmation and improvement of the contact state between the second measurement electrode 12 and the user.

Here, for example, if the controller 19 determines that the contact state between the first measurement electrode 12 and the user is good, the electroencephalogram measured by the first measurement electrode 12 and the signal resulting from the commercial power supply whose signal amplitude D is equal to or less than the predetermined threshold value are superimposed and displayed on the display screen of the display apparatus 31. Alternatively, only the electroencephalogram measured by the first measurement electrode 12 is displayed.

If only the electroencephalogram measured by the first measurement electrode 12 is displayed on the display screen of the display apparatus 31, the commercial power supply noise input from the first measurement electrode 12 side to the amplifier circuit 111 and the commercial power supply noise input from the reference electrode 14 side to the amplifier circuit 111 are canceled. This is because the contact impedance matching between the first measurement electrode 12 and the reference electrode 14 is obtained.

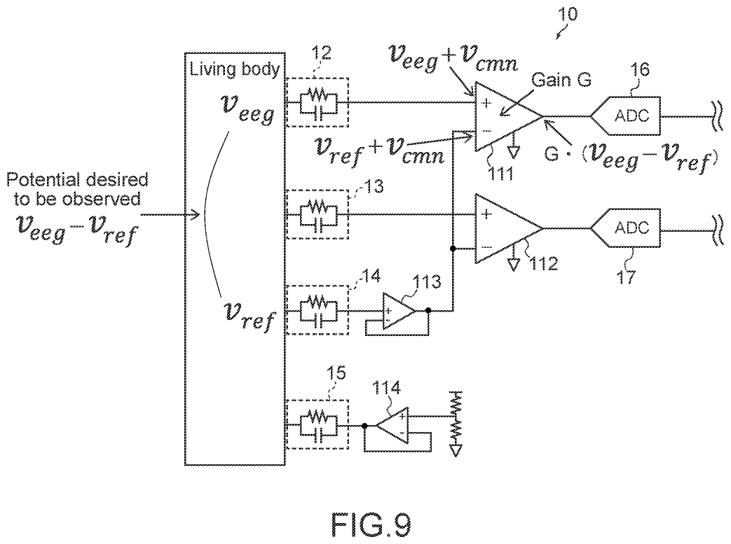

FIG. 9 is a diagram showing a detailed configuration of the biopotential measuring apparatus 10 and is a diagram for describing that the commercial power supply noise is canceled.

If the contact impedance matching between the first measurement electrode 12 and the reference electrode 14 is obtained, assuming that the potential measured by the first measurement electrode 12, the potential measured by the reference electrode 14, and the commercial power supply noise are ν_{eeg}, ν_{ref}, and ν_{cmn}, respectively, a potential P1 observed on the positive side of the amplifier circuit 111 and a potential P2 observed on the negative side of the amplifier circuit 111 are calculated by the following equations (1) and (2).

P1=ν_{eeg}+ν_{cmn} (1)

P2=ν_{ref}+ν_{cmn} (2)

Therefore, assuming that the amplifier gain of the amplifier circuit 111 is denoted by G, the amplifier circuit 111 outputs a potential P3 calculated by the following equation (3). Thus, the biopotential in which the commercial power supply noise does not remain is obtained, and thus only the electroencephalogram of the user is displayed on the display screen of the display apparatus 31 as shown in FIG. 6a.

P3=G·{(ν_{eeg}+ν_{cmn})−(ν_{ref}+ν_{cmn})}=G·(ν_{eeg}−ν_{ref}) (3)

(Pattern 4)

If the signal amplitude D of the commercial power supply noise remaining on the output side of the amplifier circuit 112 does not exceed the predetermined threshold value (NO in Step S106), the controller 19 determines that the contact state between each of the first and second measurement electrodes 12 and 13 and the user is good (Step S108).

<Functions and Effects>

Conventionally, in a biopotential measuring apparatus for measuring an electroencephalogram, an electrocardiogram, and the like, a configuration is known in which a reference potential is used as a reference and a potential difference from each measurement site is acquired via a differential amplifier circuit. In such a configuration, the measurement site is at a plurality of locations of two or more channels, the reference potential is often subjected to impedance conversion using a buffer circuit, and the signal quality of the biopotential measurement device depends greatly on the mounting state of the user and the electrode.

The mounting state of the user and the electrode can be monitored by measuring the contact impedance between the user and the electrode at each electrode. An electroencephalograph equipped with such an electrode measures an electroencephalogram and contact impedance simultaneously.

As shown in FIG. 2, when the circuit model of the contact portion between the electrode and the user has a configuration in which a resistance component and a capacitance component are connected in parallel, contact impedance is represented by, for example, the following equation (4) assuming that the contact impedance, a resistance value of the resistance component, a capacitance value of the capacitance component, an angular frequency, an imaginary unit, and a measured frequency are Z, R₁, C₁, ω, j, and f, respectively.

Z=(R₁·1/jωC₁)/(R₁+1/jωC₁)=R₁/(1+j·2πfR₁C₁) (4)

Here, in the measurement of the electroencephalogram of the user, the contact impedance in the range of DC to 60 Hz is important, but in order to simultaneously measure the electroencephalogram and the contact impedance, the electroencephalogram must be measured in the high-frequency band, and accordingly, the contact impedance must also be measured in the high-frequency band. At that time, according to the equation (4), as the measurement frequency becomes higher, the impedance of the capacitive component becomes smaller, with the result that the contact impedance is reduced.

Originally, in the biopotential measuring apparatus, the contact impedance is measured to confirm the signal quality when measuring the electroencephalogram of the user, but it may make no sense to measure the contact impedance depending on the use state of the apparatus.

For example, if the electrode is not in contact with at least the scalp of the user, the electroencephalogram cannot be measured. However, if the contact impedance is measured in the high-frequency band, the contact impedance becomes low (the signal amplitude of the contact impedance becomes small) even if the electrode comes into contact with the hair of the user, and it becomes difficult to determine whether the signal quality is good or not, which may cause an erroneous determination.

In contrast to the above, the biopotential measuring apparatus 10 of this embodiment determines the contact state of the electrode on the basis of whether the signal intensity (signal amplitude) of the commercial power supply noise (hum noise) superimposed on the biopotential waveform exceeds a predetermined threshold value. Thus, irrespective of whether the contact impedance is low or high, that is, whether the signal amplitude of the contact impedance is small or large, it is possible to accurately determine the quality of the contact state between the user and the electrode while simultaneously measuring the electroencephalogram and the contact impedance.

Further, according to the biopotential measuring apparatus 10, since the determination index for determining the contact state between the user and the electrode is the commercial power supply noise, it is possible to determine the quality of the contact state of the electrode with a simple configuration, without increasing the number of parts separately in order to determine the contact state.

<Modifications>

Although the embodiment of the present technology has been described above, the present technology is not limited to the embodiment described above, and various modifications may be made thereto.

For example, each of the amplifier circuits 111 and 112 may have one stage or may be cascaded in two or more stages. Further, for example, an analog filter block may be provided between the amplifier circuit 111, 112 and the ADC 16, 17.

Further, a plurality of predetermined threshold values may be set in the above embodiment, and the contact state between the user and the electrode may be determined in three stages such as “good”, “medium”, and “poor” in a stepwise manner. Further, the determined contact state may be displayed in color-coded form by the display apparatus 31, for example, so that the user may be prompted to explicitly improve the contact state.

Further, the biopotential measuring apparatus 10 of the above embodiment has a configuration including two electrodes (first and second measurement electrodes 12 and 13) for measuring the electroencephalogram of the user, but the present technology is not limited thereto and may have a configuration including one electrode or three or more electrodes.

<Supplement>

The embodiment of the present technology may include, for example, the biopotential measuring apparatus as described above, a biopotential measuring system, a biopotential measuring method performed in the biopotential measuring apparatus or the biopotential measuring system, a program for causing the biopotential measuring apparatus to function, and a non-transitory, tangible medium on which a program is recorded.

Further, in the embodiment described above, the description has been made on the assumption that the biopotential measuring apparatus measures the electroencephalogram of the user, but the present technology is not limited thereto. For example, the present technology may be applied to an electrocardiograph for measuring the electrocardiogram of a user, and its application is not particularly limited.

In addition, the effects described herein are illustrative or exemplary only and not restrictive. In other words, the present technology may provide other effects apparent to those skilled in the art from the description herein, in addition to or instead of the effects described above.

Although the suitable embodiment of the present technology has been described in detail above with reference to the accompanying drawings, the present technology is not limited to such an example. It is clear that persons who have common knowledge in the technical field of the present technology could conceive various alterations or modifications within the scope of the technical idea described in the Claims. It is understood that of course such alterations or modifications also fall under the technical scope of the present technology.

Note that the present technology can have the following configurations.
(1) A biopotential measuring apparatus, including:

an electrode that measures a biopotential; and

a controller that determines a quality of a contact state between the electrode and a living body on the basis of a signal amplitude of a signal of a specific frequency.
(2) The biopotential measuring apparatus according to (1), in which

the controller determines the quality of the contact state on the basis of whether the signal amplitude exceeds a predetermined threshold value.
(3) The biopotential measuring apparatus according to (1) or (2), in which

the signal of the specific frequency is a signal resulting from a commercial power supply.
(4) The biopotential measuring apparatus according to (3), further including

a reference electrode that provides a reference point of a potential at a time when a potential of the electrode is measured.
(5) The biopotential measuring apparatus according to (4), further including

an amplifier circuit that amplifies a potential difference between a potential measured by the electrode and a potential measured by the reference electrode.
(6) The biopotential measuring apparatus according to (5), in which

the electrode includes
a first measurement electrode, and
a second measurement electrode, and

the amplifier circuit includes
a first amplifier circuit connected to the first measurement electrode, and
a second amplifier circuit connected to the second measurement electrode.
(7) The biopotential measuring apparatus according to (6), in which

the controller determines that a contact state between the living body and the reference electrode is poor when each of a signal amplitude of a signal resulting from the commercial power supply and output from the first amplifier circuit and a signal amplitude of a signal resulting from the commercial power supply and output from the second amplifier circuit exceeds a predetermined threshold value.
(8) The biopotential measuring apparatus according to (6) or (7), in which

the controller determines that a contact state between the living body and the reference electrode is good when one of a signal amplitude of a signal resulting from the commercial power supply and output from the first amplifier circuit and a signal amplitude of a signal resulting from the commercial power supply and output from the second amplifier circuit does not exceed a predetermined threshold value.
(9) The biopotential measuring apparatus according to any one of (6) to (8), in which

the controller determines that a contact state between the first measurement electrode and the living body is poor and that a contact state between the second measurement electrode and the living body is good when the signal amplitude of the signal resulting from the commercial power supply and output from the first amplifier circuit exceeds the predetermined threshold value.
(10) The biopotential measuring apparatus according to any one of (6) to (9), in which

the controller determines that the contact state between the first measurement electrode and the living body is good and that the contact state between the second measurement electrode and the living body is poor when the signal amplitude of the signal resulting from the commercial power supply and output from the first amplifier circuit does not exceed the predetermined threshold value and when the signal amplitude of the signal resulting from the commercial power supply and output from the second amplifier circuit exceeds the predetermined threshold value.
(11) The biopotential measuring apparatus according to any one of (6) to (10), in which

the controller determines that the contact state between each of the first and second measurement electrodes and the living body is good when each of the signal amplitude of the signal resulting from the commercial power supply and output from the first amplifier circuit and the signal amplitude of the signal resulting from the commercial power supply and output from the second amplifier circuit does not exceed the predetermined threshold value.
(12) The biopotential measuring apparatus according to any one of (1) to (11), in which

the controller causes a display apparatus to display information for making a user confirm the contact state when determining that the contact state is poor.
(13) The biopotential measuring apparatus according to any one of (1) to (12), in which

the biopotential measuring apparatus is configured to be capable of measuring an electroencephalogram.
(14) The biopotential measuring apparatus according to any one of (1) to (13), in which

the biopotential measuring apparatus is configured to be capable of measuring an electrocardiogram.
(15) A biopotential measuring system, including:

a biopotential measuring apparatus including
an electrode that measures a biopotential, and
a controller that determines a quality of a contact state between the electrode and a living body on the basis of a signal amplitude of a signal of a specific frequency; and

a display apparatus that displays the biopotential and the contact state between the electrode and the living body.
(16) The biopotential measuring system according to (15), in which

the display apparatus displays information for making a user confirm the contact state when the controller determines that the contact state is poor.
(17) A biopotential measuring method, including:

by a biopotential measuring apparatus,

measuring a biopotential; and

determining a quality of a contact state between the electrode and a living body on the basis of a signal amplitude of a signal of a specific frequency.

### REFERENCE SIGNS LIST

biopotential measuring system 1
biopotential measuring apparatus 10
differential amplifier circuit (amplifier circuit) 11
first measurement electrode 12
second measurement electrode 13
reference electrode 14
ADC 16, 17
controller 19
information processing apparatus 30, 100
display apparatus 31
amplifier circuit 111 (first amplifier circuit), 112 (second amplifier circuit)
impedance conversion circuit 113, 114

1. A biopotential measuring apparatus, comprising:
an electrode that measures a biopotential; and
a controller that determines a quality of a contact state between the electrode and a living body on a basis of a signal amplitude of a signal of a specific frequency.

2. The biopotential measuring apparatus according to claim 1, wherein
the controller determines the quality of the contact state on a basis of whether the signal amplitude exceeds a predetermined threshold value.

3. The biopotential measuring apparatus according to claim 1, wherein
the signal of the specific frequency is a signal resulting from a commercial power supply.

4. The biopotential measuring apparatus according to claim 3, further comprising
a reference electrode that provides a reference point of a potential at a time when a potential of the electrode is measured.

5. The biopotential measuring apparatus according to claim 4, further comprising
an amplifier circuit that amplifies a potential difference between a potential measured by the electrode and a potential measured by the reference electrode.

6. The biopotential measuring apparatus according to claim 5, wherein
the electrode includes
a first measurement electrode, and
a second measurement electrode, and
the amplifier circuit includes
a first amplifier circuit connected to the first measurement electrode, and
a second amplifier circuit connected to the second measurement electrode.

7. The biopotential measuring apparatus according to claim 6, wherein
the controller determines that a contact state between the living body and the reference electrode is poor when each of a signal amplitude of a signal resulting from the commercial power supply and output from the first amplifier circuit and a signal amplitude of a signal resulting from the commercial power supply and output from the second amplifier circuit exceeds a predetermined threshold value.

8. The biopotential measuring apparatus according to claim 6, wherein
the controller determines that a contact state between the living body and the reference electrode is good when one of a signal amplitude of a signal resulting from the commercial power supply and output from the first amplifier circuit and a signal amplitude of a signal resulting from the commercial power supply and output from the second amplifier circuit does not exceed a predetermined threshold value.

9. The biopotential measuring apparatus according to claim 8, wherein
the controller determines that a contact state between the first measurement electrode and the living body is poor and that a contact state between the second measurement electrode and the living body is good when the signal amplitude of the signal resulting from the commercial power supply and output from the first amplifier circuit exceeds the predetermined threshold value.

10. The biopotential measuring apparatus according to claim 8, wherein
the controller determines that the contact state between the first measurement electrode and the living body is good and that the contact state between the second measurement electrode and the living body is poor when the signal amplitude of the signal resulting from the commercial power supply and output from the first amplifier circuit does not exceed the predetermined threshold value and when the signal amplitude of the signal resulting from the commercial power supply and output from the second amplifier circuit exceeds the predetermined threshold value.

11. The biopotential measuring apparatus according to claim 8, wherein
the controller determines that the contact state between each of the first and second measurement electrodes and the living body is good when each of the signal amplitude of the signal resulting from the commercial power supply and output from the first amplifier circuit and the signal amplitude of the signal resulting from the commercial power supply and output from the second amplifier circuit does not exceed the predetermined threshold value.

12. The biopotential measuring apparatus according to claim 1, wherein
the controller causes a display apparatus to display information for making a user confirm the contact state when determining that the contact state is poor.

13. The biopotential measuring apparatus according to claim 1, wherein
the biopotential measuring apparatus is configured to be capable of measuring an electroencephalogram.

14. The biopotential measuring apparatus according to claim 1, wherein
the biopotential measuring apparatus is configured to be capable of measuring an electrocardiogram.

15. A biopotential measuring system, comprising:
a biopotential measuring apparatus including
an electrode that measures a biopotential, and
a controller that determines a quality of a contact state between the electrode and a living body on a basis of a signal amplitude of a signal of a specific frequency; and
a display apparatus that displays the biopotential and the contact state between the electrode and the living body.

16. The biopotential measuring system according to claim 15, wherein
the display apparatus displays information for making a user confirm the contact state when the controller determines that the contact state is poor.

17. A biopotential measuring method, comprising:
by a biopotential measuring apparatus,
measuring a biopotential; and
determining a quality of a contact state between the electrode and a living body on a basis of a signal amplitude of a signal of a specific frequency.
